(19) United States
(12) Patent Application Publication
(10) Pub. No.: US 2018/0264043 A1
(43) Pub. Date: Sep. 20, 2018

(54) RESTORATION OF DETERIORATED TISSUE IN THE FACE OR SELECTED AREAS OF THE BODY WITH MESENCHYMAL STEM CELLS

(71) Applicant: HOLDING PEN, LLC, Berthoud, CO (US)

(72) Inventors: Kenneth Allen Pettine, Fort Collins, CO (US); Gale Kennedy, Berthoud, CO (US)

(21) Appl. No.: 15/458,853

(22) Filed: Mar. 14, 2017

(51) Int. Cl. A61K 35/28; A61K 9/00; A61K 35/35; A61K 47/26; A61K 47/02; A61K 35/19; A61M 1/00; A61M 5/32; A61M 37/00; A61M 5/30

(52) U.S. Cl. CPC A61K 35/28; A61K 9/0019; A61K 35/35; A61K 47/26; A61K 47/02; A61K 2035/124; A61K 35/19; A61M 1/0023; A61M 5/3298; A61M 37/0015; A61M 5/30; A61K 9/0021

(57) ABSTRACT

The present disclosure provides an inventive method of treating deteriorated skin tissue in the face or selected areas of the body, wherein the skin tissue has deteriorated due to aging and/or other factors, by injecting mesenchymal stem cells (MSCs) into these areas, resulting in improving and/or restoring the affected areas. In one aspect, the MSCs are obtained autogenously. In another aspect, the MSCs are minimally manipulated. In yet another aspect, treatment of the skin tissue is carried out using bone marrow concentrate that contains the MSCs. A platelet rich plasma (PRP) may be added to a bone marrow concentrate containing MSCs.

### BACKGROUND OF THE INVENTION

Skin is composed of the epidermis and the dermis. Below these layers lies the hypodermis, which is not usually classified as a layer of skin. The hypodermis is also commonly referred to as subcutaneous fat layer, or subcutaneous tissue. The outermost epidermis is made up of stratified squamous epithelium with an underlying basement membrane. The outermost epidermis contains no blood vessels, and is nourished by diffusion from the dermis. The main type of cells which make up the epidermis are keratinocytes. Also present are melanocytes and langerhans cells. This layer of skin is responsible for keeping water in the body and keeping harmful chemicals and pathogens out.

The dermis lies below the epidermis and contains a number of structures including blood vessels, nerves, hair follicles, smooth muscle, glands and lymphatic tissue. The dermis (or corium) may be 3-5 mm thick and is the major component of human skin. The dermis is composed of a network of connective tissue, predominantly collagen fibrils providing support and elastic tissue providing flexibility. The main cell types are fibroblasts, adipocytes and macrophages. The hypodermis lies below the dermis. The hypodermis' purpose is to attach the skin to underlying bone and muscle as well as supplying the skin with blood vessels and nerves. The hypodermis is made up of loose connective tissue and elastin. The main cell types are fibroblasts, macrophages and adipocytes. The hypodermis contains 50% of body fat. Fat serves as padding and insulation for the body.

### SUMMARY OF THE INVENTION

An embodiment of the invention may therefore comprise a treatment method for improving or restoring deteriorated skin tissue in the face or selected areas of the body of a person, comprising obtaining mesenchymal stem cells from the person, and using a needle matrix to inject of the mesenchymal stem cells into the skin tissue, the mesenchymal stem cells being one of autogenous mesenchymal stem cells and allogeneic stem cells, wherein the mesenchymal stem cells improve or restore the skin tissue.

An embodiment of the invention may further comprise a method for treating deteriorated skin tissue in the face or selected areas of the body of a person with mesenchymal stem cells, comprising aspirating autogenous or allogeneic bone marrow to obtain bone marrow aspirate containing the mesenchymal stem cells, concentrating the bone marrow aspirate to obtain bone marrow concentrate, the bone marrow concentrate containing the mesenchymal stem cells, and using a needle matrix to inject the bone marrow concentrate into the deteriorated skin tissue, wherein the mesenchymal stem cells improve or restore the skin tissue.

An embodiment of the invention may further comprise a method of treatment of skin tissue, the method comprising obtaining mesenchymal stem cells from a person, and injecting the mesenchymal stem cells into the skin tissue with an injector using a matrix needle injector.

An embodiment of the invention may further comprise a v method of treatment of skin tissue, the method comprising obtaining autogenous or allogeneic mesenchymal stem cells and injecting the mesenchymal stem cells into the skin tissue using a needle free injector.

An embodiment of the invention may further comprise an apparatus for treating skin tissue, the apparatus comprising a reservoir that supplies a fluid, a micro needle matrix connected to the reservoir; and an injector that pushes the fluid through the micro needle matrix and into the skin tissue.

An embodiment of the invention may further comprise a method for treating deteriorated skin tissue in the face or selected areas of the body of a person with mesenchymal stem cells, comprising aspirating bone marrow to obtain bone marrow aspirate containing the mesenchymal stem cells, concentrating the bone marrow aspirate to obtain bone marrow concentrate, the bone marrow concentrate containing the mesenchymal stem cells, collecting blood, centrifuging the blood to separate platelets in the blood, concentrating the platelets to obtain a platelet rich plasma, mixing the concentration of platelets with the concentration of bone marrow aspirate containing the bone marrow concentrate, and using a micro needle matrix to inject the mixture of the concentration of platelets and the bone marrow concentrate into the deteriorated skin tissue, wherein the mixture of the concentration of platelets with the concentration of bone marrow aspirate containing the bone marrow concentrate containing the mesenchymal stem cells acts to produce more fibroblasts in the deteriorated skin and stimulate the fibroblasts in the deteriorated skin.

An embodiment of the invention may further comprise a method for treating deteriorated skin tissue in the face or selected areas of the body of a person with mesenchymal stem cells, comprising obtaining an autogenous or allogeneic SVF (stromal vascular fraction) from adipose, concentrating the SVF to obtain adipose concentrate, the adipose concentrate containing the mesenchymal stem cells, and using a needle matrix to inject adipose concentrate into the deteriorated skin tissue, wherein the mesenchymal stem cells improve or restore the skin tissue.

An embodiment of the invention may further comprise a method for treating deteriorated skin tissue in the face or selected areas of the body of a person with mesenchymal stem cells, comprising obtaining an SVF (stromal vascular fraction) from adipose, concentrating the SVF to obtain adipose concentrate, the adipose concentrate containing the mesenchymal stem cells, collecting blood, centrifuging the blood to separate platelets in the blood, concentrating the platelets to obtain a platelet rich plasma, mixing the concentration of platelets with the adipose concentrate containing the mesenchymal stem cells, and using a micro needle matrix to inject the mixture of a concentration of platelets and the adipose concentrate into the deteriorated skin tissue, wherein the mixture of the concentration of platelets with adipose concentrate containing the mesenchymal stem cells acts to produce more fibroblasts in the deteriorated skin and stimulate the fibroblasts in the deteriorated skin.

An embodiment of the invention may further comprise an apparatus for treating skin tissue, the apparatus comprising a reservoir that supplies a fluid to a matrix of needles, the reservoir containing a mixture comprising mesenchymal stem cells and a platelet rich plasma a needle matrix connected to the reservoir, and an injector that pushes the fluid through the needle matrix into the skin tissue.

### BRIEF DESCRIPTION OF THE DRAWINGS

FIG. 1 is an illustration of an embodiment of the pelvic girdle showing a needle advanced through the iliac crest and into bone marrow to aspirate bone marrow from the ilium.

FIG. 2 is an illustration of an embodiment of a micro needling injector for injection of mesenchymal cells.

FIG. 3 is an illustration of an embodiment of a liquid injector for injection of mesenchymal cells.

FIG. 4 is an illustration of an embodiment of flow diagram showing a method of the invention.

FIG. 5A shows an illustration of an embodiment of a round needle injector.

FIG. 5B shows an illustration of an embodiment of a rectangular needle injector.

FIG. 5C shows an illustration of an embodiment of an adjustable needle injector 465.

FIG. 6 shows an illustration of an embodiment of a pouch injector.

FIG. 7 shows an illustration of an embodiment of a pouchless squeeze injector.

FIG. 8 shows an illustration of an embodiment of a flow diagram showing a method of the invention

### DETAILED DESCRIPTION OF THE PREFERRED EMBODIMENTS

An embodiment of the disclosed invention provides formulations and methods for treating skin tissue using autogenous or allogeneic mesenchymal stem cells (MSC) derived from bone marrow or adipose tissue. In one aspect, the MSCs can be utilized from bone marrow concentrate (BMC) or from adipose stromal vascular fraction (SVF). In another aspect, the MSCs can also be utilized after cell expansion. It is intended here that the term autogenous is meant to convey a sample originated within the body of the particular patient within which any treatment is used, and may include autologous samples and treatments.

Skin aging includes chronological aging as well as photoaging, and may appear as wrinkling, lack of elasticity (for example sagging), uneven pigmentation, thinning of the skin and/or collagen so that veins and other underlying structures become more prominent, and the like. Skin aging is a major example of a skin condition that involves a decrease in cell proliferation and in cell function. As used herein, “skin aging” refers to alterations in the appearance and function of skin that occur with aging, such as wrinkling, loss of elasticity, sagging, uneven pigmentation (for example, “age spots” or “liver spots”), and loss of underlying tissue mass. Such conditions may be accelerated and/or exacerbated by exposure to ultraviolet radiation (“photoaging”) and other environmental conditions. The terms “autogenous” and “autologous” may be used interchangeably throughout this application.

Aging may cause deterioration of facial and other skin. Facial aging may occur as the result of several factors: inherent changes within the skin, effects of gravity, facial muscles acting on the skin (dynamic lines), soft tissue loss or shift and bone loss and loss of tissue elasticity. The skin ages when the epidermis begins to thin, causing the junction with the dermis to flatten. Collagen may decrease as a person ages and the bundles of collagen, which give the skin turgor, may become looser and lose strength. When the skin loses elasticity, it is less able to resist stretching. Coupled with gravity, muscle pull and tissue changes, the skin begins to wrinkle. Water loss and breakdown of bonds between cells also reduces the barrier function of the skin, which may cause the skin's pore size to increase.

As a person ages, the face loses volume, soft tissue, and fat. The appearance of jowls and folds is usually caused by the drooping of facial tissues and folding of areas where the muscles below are attached to the skin. As part of the reduction in soft tissue the face gets more hollow.

More specifically, in various facial areas, such as forehead, eyes, nose, midface and lower face, changes relating to aging have been well documented. In the forehead area, the forehead and brow droop over time, which lowers the eyebrows and causes the upper eyelid skin to bunch. Forehead lines appear when one tries to hold the brows and eyelids up to counteract these changes. Eyes may be the first facial feature to show signs of aging. Skin changes around the eyes may occur earlier than in the rest of the face since the skin is thinner around the eyes. The skin around the eyes contains fewer glands and is subjected to constant blinking, squinting, rubbing, and pulling. The midface may age when the cheeks begin to droop, causing nasolabial folds. Nasolabial folds are the lines that run from the sides of the nose to the corners of the mouth. In the nose area, as a person ages, the nose may elongate. Elongation may result from thinning of the soft tissue and loss of elasticity, which causes “drooping of the tip” and unmasking of the bone, creating a new hump. In the lower face area, as the face ages, facial tissues may descend. Folds and lines may result in this area. Further down on the face, the corners of the mouth may droop, and descent of the jowls may create folds often referred to as “marionette” lines. Furthermore, jowls may form when the cheeks sag around a fixed point along the jaw where the facial muscles attach to the jawbone. The facial muscles continue down into the neck as a sheet called the platysma muscle. This muscle may gap in the center of the neck, creating two bands.

Various injectables may be used for restoring tissue loss in the face. Injectable collagen may be used as a soft-tissue filler to fill wrinkles, lines and scars on the face. Collagen is a naturally occurring protein that supports various parts of the body including skin, tendons and ligaments. Fat injections may be used for years to add volume, fill wrinkles and lines, and enhance the lips. Fat injections involve taking fat from one part of the patient's body (abdomen, thighs or buttocks) and reinjecting it beneath the facial skin. Botulinum toxins may be used for neck spasms, cranial nerve disorders and eye spasms. Botox may be used for cosmetic use in the glabellar region, and may be used to smooth wrinkles. When injected into facial muscles botulinum toxins block nerve impulses, temporarily paralyzing muscles and smoothing wrinkles.

All publications cited throughout this application, are incorporated herein by reference in their entirety.

Some researchers have reported on skin revitalization using injections of platelet-rich plasma. For example, Redaelli, A., et al. (“Face and Neck Revitalization with Platelet-Rich Plasma (PRP): Clinical Outcome in a Series of 23 Consecutively Treated Patients,” Journal of Drugs in Dermatology, 9(5):466-471 (2010)) describe limited success in revitalizing facial and neck skin with injections of platelet-rich plasma (PRP), activated with calcium chloride, at standard injection points, into the face and neck skin.

Hyaluronic acid is one of the most commonly used cosmetic dermal fillers which adds volume to minimize wrinkles and lines. Hyaluronic acid is a linear polysaccharide that exists naturally in all living organisms and is a universal component of the extra-cellular spaces of body tissues. The identical structure of hyaluronic acid in all species and tissues makes this polysaccharide an ideal substance for use as a bio-material in health and medicine. Hyaluronic acid is present in many places in the human body. It gives volume to the skin, shape to the eyes and elasticity to the joints. The highest concentrations are found in connective tissues, and most hyaluronic acid (about 56%) is found in the skin. Various forms of hyaluronic acid are provided commercially by several manufacturers. The most commonly used hyaluronic acid is the non-animal stabilized hyaluronic acid (NASHA) in a clear gel form, produced by bacterial fermentation from streptococci bacteria. Different from animal derived hyaluronic acid, the non-animal derived hyaluronic acid is free from animal proteins. This is believed to limit the risk of animal based disease transmissions or development of allergic reactions to animal proteins.

Although hyaluronic acid and derivatives are the most commonly used dermal fillers, they have limited viability. It is reported that re-injection is needed every 4 to 12 months, or even more frequently.

In recent years, other researchers have reported on the use of compositions of hyaluronic acid that comprise stem cells and a variety of additional components. See for example, Giampapa, V., et al. (“Method and Composition for Restoration of Age-Related Tissue Loss in the Face or Selected Areas of the Body,” U.S. patent application Ser. No. 13/318,524 (May 4, 2010) (abandoned)) which discloses an injectable composition and a method of its use to restore skin. The composition includes stem cells (e.g., peripheral blood stem cells, bone marrow-derived blood stem cells, or mesenchymal stem cells) and hyaluronic acid as a carrier, as well as growth factors such as insulin, an insulin-like growth factor, a thyroid hormone, a fibroblast growth factor, an estrogen, and retinoic acid. Further, the composition may include adipocytes. Additionally, the method includes carrying out a micro-abrasion therapy step prior to injection. Thus, the skin to be treated is prepared by performing microdermabrasion, presumably to promote cellular repair, which creates an environment that allows the injected stem cells to thrive.

Although various injectables may be used clinically for restoration of tissue loss in the face over the past half century, due to various limitations in the materials or compatibility with the tissues, the long-term effect in restoring the tissue loss is limited. Injectables and treatment methods to enhance the overall effects in restoration of tissue loss in the face or selected areas of the body, such as the neck and hands are disclosed herein.

The use of stem cells and stem cell derivatives may provide reagents for treating tissue damage as a result of aging, genetic defects, injuries (such as burns, for example), and/or disease processes. Ideally, cells that are capable of differentiating into the affected cell types could be introduced into a subject in need thereof, where they would interact with the organ microenvironment and supply the necessary cell types to repair the tissue damage.

For the purposes of this disclosure, MSCs may be derived from multiple sources including, bone marrow, adipose, blood, dermis, periosteum and tissues. In various embodiments, MSCs are extracted from patient intended to receive the cellular therapy (i.e., an autologous transplant). MSCs can be culture expanded prior to their introduction into the patient (those skilled in the art will understand the techniques and methodologies for cell expansion). Growth of MSCs in vitro can be used, for example, to increase the number of MSCs available for implantation or injection. In non-limiting examples, MSC numbers are increased about two-fold or greater, about ten-fold or greater, or about twenty-fold or greater or more, depending on the desired number of cells. Patient-specific master cell banks, for example can include ten or more passages of expanded autologous cells, which increases MSC counts by many orders of magnitude in comparison to primary isolates. In various embodiments, growing MSCs in vitro can include expansion in a cell culture medium, which includes nutrients, buffers, salts, proteins, vitamins and/or growth factors, which promote MSC growth. A useful cell culture medium is RPMI 1680 supplemented with 10% serum, and appropriate antibiotics such as penicillin/streptomycin and G418. Human serum is preferred for expanding the MSCs for human transplant preparations, but fetal bovine serum has produced acceptable yields of the MSCs in culture. A currently preferred culture system is an environmentally controlled, closed-culture system, commonly known as a bioreactor, which is particularly useful for collection of MSC-conditioned media. Other systems are suitable for culture of MSCs, the expansion of donor cells, and the creation of master MSC banks. These should comply with Good Tissue Practices and ideally should be cGMP. The expanded cells are commonly but not necessarily frozen prior to implantation, and a suitable cryogenic medium that is acceptable to the FDA for such purposes is CryoStorCSlO, available from BioLife Solutions, Bothell, Wash.

Mesenchymal stem cells (MSCs), after their initial discovery in bone marrow, have been isolated and characterized from several adult and fetal tissues, including adipose (fat), dermis (skin), synovial fluid, periosteum, umbilical cord blood, placenta and amniotic fluid. MSCs are partially defined by their ability to differentiate into tissues including osteoblasts (bone cells), chondrocytes (cartilage cells), myocytes (muscle cells), and adipocytes (fat cells). But it is believed that it is their trophic, paracrine and immunomodulatory functions that may have the greatest therapeutic impact in vivo. Unlike pharmaceutical treatments that deliver a single agent at a specific dose, MSCs are site regulated and secrete bioactive factors and signals at variable concentrations in response to local microenvironmental cues. Significant progress has been made in understanding the biochemical and metabolic mechanisms and feedback associated with MSC response. The anti-inflammatory and immunomodulatory capacity of MSC may be paramount in the restoration of localized or systemic conditions for normal healing and tissue regeneration. Allogeneic MSC treatments, categorized as a drug by regulatory agencies, have been widely pursued, but new studies demonstrate the efficacy of autologous MSC therapies, even for individuals affected by a disease state. Safety and regulatory concerns surrounding allogeneic cell preparations make autologous and minimally manipulated cell therapies an attractive option for many regenerative, anti-inflammatory and autoimmune applications. (See for example, Murphy, M. B., et al., “Mesenchymal stem cells: environmentally responsive therapeutics for regenerative medicine,” Experimental & Molecular Medicine, 45:e54 (2013)).

The primary trophic property of MSCs is the secretion of growth factors and other chemokines to induce cell proliferation and angiogenesis. MSCs express mitogenic proteins such as transforming growth factor-alpha (TGF-α), TGF-ß, hepatocyte growth factor (HGF), epithelial growth factor (EGF), basic fibroblast growth factor (FGF-2) and insulin-like growth factor-1 (IGF-1) to increase fibroblast, epithelial and endothelial cell division. Vascular endothelial growth factor (VEGF), IGF-1, EGF and angiopoietin-1 are released to recruit endothelial lineage cells and initiate vascularization. It has been hypothesized that an individual's genotype has a role in the expression of and reaction to these cytokines, providing credence to the philosophy of personalized medicine utilizing responsive agents (that is, MSCs) rather than a dose of recombinant proteins or autologous growth factors (for example, platelet-rich plasma). The trophic effects extend beyond cell proliferation to the reduction of scar tissue formation presumably by local cells secreting paracrine factors keratinocyte growth factor, stromal cell-derived factor-1 (SDF-1) and macrophage inflammatory protein-1 alpha and beta.

MSCs have been reported to possess anti-inflammatory and immunomodulatory properties. In many types of musculoskeletal trauma, inflammatory conditions at the site of injury impede the natural repair processes by local progenitor and mature cells. Without being bound by theory, it is believed that MSCs assist via paracrine mechanisms and modulate the regenerative environment via anti-inflammatory and immunomodulatory mechanisms. In response to inflammatory molecules such as interleukin-1 (IL-1), IL-2, IL-12, tumor necrosis factor-α (TNF-α) and interferon-gamma (INF-γ), MSCs secrete an array of growth factors and anti-inflammatory proteins with complex feedback mechanisms among the many types of immune cells. The key immunomodulatory cytokines include prostaglandin 2, TGF-ß1, HGF, SDF-1, nitrous oxide, indoleamine 2,3-dioxygenase, IL-4, IL-6, IL-10, IL-1 receptor antagonist and soluble tumor necrosis factor-α receptor. MSCs prevent proliferation and function of many inflammatory immune cells, including T cells, natural killer cells, B cells, monocytes, macrophages and dendritic cells. Although MSCs across species are able to regulate T-cell activity, the mechanisms are not identical across mammalian species.

A characteristic of chronically inflamed environments is a persistent imbalance in the types of helper T cells and macrophages. MSCs indirectly promote the transition of TH1 to TH2 cells by reducing INF-g and increasing IL-4 and IL-10. The restored TH1/TH2 balance has been shown to improve tissue regeneration in cartilage, muscle and other soft tissue injuries, alleviate symptoms of autoimmune diseases and have an anti-diabetic effect. Similarly, reduction in INF-γ and secretion of IL-4 promotes a shift in macrophages from M1 (pro-inflammatory, anti-angiogenic and tissue growth inhibition) to M2 (anti-inflammatory, pro-remodeling and tissue healing) type, an effect required for skeletal, muscular and neural healing and regeneration. (See, Murphy, M. B., et al., “Mesenchymal stem cells: environmentally responsive therapeutics for regenerative medicine,” Experimental & Molecular Medicine, 45:e54 (2013)).

Throughout this description, including the foregoing description of related art and cited publications, as well as any and all publications cited in what follows below, it is to be understood that any and all publicly available documents described herein, including any and all cited U.S. patents and patent applications, are specifically incorporated by reference herein in their entirety for all that they disclose or teach. Nonetheless, the related art and publications described herein are not intended in any way as an admission that any of the documents described therein, including pending U.S. patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

In one embodiment of the invention, the present application discloses an inventive method of treating deteriorated skin tissue in the face or selected areas of the body (sometimes referred to hereinafter by the term “affected areas”), wherein the skin tissue has deteriorated due to aging and/or other factors, by injecting mesenchymal stem cells (MSCs) into these areas, resulting in improving and/or restoring the affected areas. The mesenchymal stem cells are obtained autogenously, or are allogeneic stem cells. In another key aspect, the mesenchymal stem cells are minimally manipulated. In yet another key aspect, the minimally manipulated mesenchymal stem cells are not cultured. In yet another key aspect, the autogenous, minimally manipulated mesenchymal stem cells are used for treatment of a patient within a relatively short period of time, illustratively, within less than one hour of obtention from the patient.

It is to be understood that, as contemplated herein, terms such as “deteriorated skin tissue”, and similar terms used herein, refer to skin tissue conditions resulting from deterioration in the manner described herein. Illustratively, such terms include, but are not limited to, sagging skin, wrinkles and lines, tissue loss and other visible signs of aging in the skin, and the like. However, it should also be understood that these terms may apply to other related skin tissue conditions not described herein, but that are well understood by those skilled in the art to be closely related conditions. Likewise, it is to be understood that, as contemplated herein, terms such as “improvement and/or restoration” of skin tissue, and similar terms used herein, refer to partial or complete reversal of the deterioration and to partial or complete restoration of the skin tissue to normal physiologic structure. Those skilled in the art will understand that a normal physiologic structure is one that may be found in a typical 20 year old person, for example. One example of such improvement and/or restoration, in the case of facial treatment, is the commonly used term “facelift.”

Accordingly, particular embodiments of the invention herein may provide a method for treating deteriorated skin tissue resulting in improvement and/or restoration of the skin tissue. The method includes obtaining MSCs, preferably autogenously (but also allogeneically); minimally manipulating the MSCs; injecting the minimally manipulated MSCs into the affected areas of the skin; and improving and/or restoring the affected areas of the skin.

Particular embodiments of the invention may also provide a method for treating deteriorated skin tissue resulting in improvement and/or restoration of the skin tissue. The method includes obtaining bone marrow concentrate (BMC), preferably autogenously, which contains MSCs, minimally manipulate the BMC, and injecting the minimally manipulated BMC containing the MSCs into one or more of the affected areas of the skin.

Particular embodiments of the invention may also improve and/or restore affected areas of the skin. The method includes injecting minimally manipulated BMC, preferably obtained autogenously, which contains MSCs, into one or more of the affected areas of the skin.

Embodiments of the invention, disclosed herein may also include a method of treating deteriorated skin tissue resulting in improvement and/or restoration of the skin tissue. The method includes injecting uncultured MSCs into one or more locations in the affected areas of the skin tissue. In one aspect, the method comprises the use of autogenous BMC that contains the MSCs. In another aspect, the method comprises injecting the BMC at various depths into the affected areas of the skin tissue.

It is to be understood that, as contemplated herein, any suitable biologic MSC product may be utilized in the various embodiments of the invention disclosed herein. In a preferred embodiment, the biologic MSC product is BMC, but may also include SVF. BMC is an excellent source of MCSs, because the MSCs are stored in bone marrow. Illustrative examples of other sources of MSCs are peripheral blood, synovium, periosteum, skeletal muscle, and adipose tissue, and any other source of MSCs known to those skilled in the art.

MSCs obtained from BMC have been reported to possess many positive attributes. MSCs are believed to be anti-inflammatory, secrete numerous growth factors, stimulate blood vessel formation, modulate the immune system to enhance healing, fight bacteria, turn into localized cells, and potentially heal inflammation. Accordingly, in one broad embodiment, disclosed herein is a method of treating deteriorated skin tissue, wherein the skin deterioration is associated with inflammation in the skin tissue, resulting in reduction of the inflammation and in improvement and/or restoration of the skin tissue.

MSCs are the preferred cells for the purpose of this invention. MSCs have been shown to have the potential to differentiate into several lineages including bone (Haynesworth et al. (1992) 13 Bone 81-88), cartilage (Mackay et al. (1998) 4 Tissue Eng 41 5-28; Yoo et al. (1998) 80 J Bone Joint Surg Am 745-57), adipose tissue (Pittenger et al. (2000) 251 Curr Top Microbiol Immunol-11), tendon (Young et al. (1998) 16 J Orthop Res 406-13), muscle, and stroma (Caplan et al. (2001) 7 Trends Mol Med 259-64). Nonetheless, as contemplated herein, it should be understood that a variety of stem cells other than MSCs may be used.

In another embodiment of the invention, disclosed herein is a method of treating deteriorated skin tissue with autogenous MSCs resulting in improvement and/or restoration of the skin tissue. In one aspect, this method does not include nor require inclusion of commonly used cosmetic dermal fillers and carriers such as hyaluronic acid and the like. In another aspect, this method does not include nor require the addition of growth factors such as insulin, insulin-like growth factor, a thyroid hormone, a fibroblast growth factor, an estrogen, retinoic acid, and the like. In yet another aspect, this method does not include nor require the addition of adipocytes. In a further aspect, this method does not include nor require the inclusion of a step of preparing the skin by microdermabrasion or similar skin therapy to promote cellular repair. Nonetheless, as contemplated herein, it is understood that there is nothing in the disclosed invention that would prevent the use in the invention of any of the additives or steps listed in this paragraph, if desired.

In another embodiment of the invention, disclosed herein is a method of treating deteriorated skin tissue with autogenous MSCs resulting in improvement and/or restoration of the skin tissue. In one aspect, this method does not include nor require inclusion of a step of culturing and/or culture-expanding the MSCs.

In another embodiment of the invention, disclosed herein is a method of treating deteriorated skin tissue in one or more areas of the body with autogenous MSCs, resulting in improvement and/or restoration of the skin tissue. Illustratively, the one or more areas that may be treated include, but are not limited to, one or more areas of the face such as the peri-orbital area, the lips, the malar area, the nasolabial folds, and the labio-mandibular folds, and other areas of the body such as the neck and the hands.

In another embodiment of the invention, disclosed is a method for treating skin tissue by using autogenous mesenchymal stem cells. The method comprises: (a) obtaining autogenous mesenchymal stem cells; (b) applying said autogenous mesenchymal stem cells onto said skin tissue; (c) rejuvenating, augmenting, and/or repairing said skin tissue. In one aspect, the method further comprises obtaining autogenous bone marrow concentrate containing said autogenous mesenchymal stem cells. The method further comprises aspirating autogenous bone marrow or suctioning adipose tissue to obtain said autogenous bone marrow concentrate, or SVF. Illustratively, the method further comprises aspirating said autogenous bone marrow to obtain about 5 milliliters to about 55 milliliters of said autogenous bone marrow concentrate. The method further comprises aspirating autogenous bone marrow of an ilium of a pelvis. The method further comprises minimally manipulating the autogenous bone marrow by processing it as described in Pettine, K. A., U.S. Pat. No. 9,408,874 (Aug. 9, 2016), which is incorporated herein by reference in its entirety for all that it discloses or teaches.

BMA (bone marrow aspirate) and BMC (bone marrow concentrate) are known to contain hematopoietic as well as MSC populations. The method of extraction is typically correlative to the source of these cells. Typically, the novel technology utilizes an approach, whereby approximately 10 cc of BMA is drawn with typically frequent rotation and repositioning. Additional draws are done after deeper placement of a needle in the iliac crest.

Bone marrow collection and processing to BMC is carried out herein as described in Pettine, K. A., U.S. Pat. No. 9,408,874 (Aug. 9, 2016), which is incorporated herein by reference in its entirety for all that it discloses or teaches. This provides a pre-determined amount of a processed bone marrow concentrate with a pre-determined amount of a pre-mixture, where the pre-mixture includes quantities of anticoagulant solution, dextrose and phosphate buffered saline. Typically, the iliac crest, and more typically the posterior iliac crest, is where bone marrow aspirate may be harvested in a surgical setting, however any suitable area where BMA may be extracted may be used. The novel cellular “snapshot’ is derived via timely methodologies specific to non-expanded, minimally manipulated, autologous cell and associated endogenous microenvironment (“milieu”). Typically, once extracted, the BMA and the resulting BMC is obtained by isolation of the desired cell populations via centrifugation. However, any suitable means may be used to obtain the BMC from the BMA. When centrifugation is used, the BMA is separated according to the slightly differing specific gravities of the aspirated cell types. The cells contained in the BMA can be stratified under centrifugation. The volume, rate, and time of centrifugation are important for controlling the resulting biologic factors contained within the endogenous milieu. Typically, the longer the processing time and/or the more agitation and handling, the lower the oxygen level in the extracted cells which typically include cellular constituents and components sequestered in the residual endogenous milieu, wherein the milieu typically includes antigens, surface biomarkers, proteins and growth factors for angiogenesis, osteogenesis, other regenerative outcomes, and the like. In some embodiments, the degradative manipulation and resultant influence during processing is limited. The resulting, unadulterated milieu may retain a large number of unchanged biologic drivers, markers and signals that are dose appropriate and specific to the cascade of healing found through the native physiology. The stratification and selection of MSCs and progenitors from this population may influence traits such as specific plasticity and immunomodulation. Typically, the time between extraction and re-implantation or re-injection is within 1 hour, and more typically within 30 minutes, and still more typically within 20 minutes. Thus, a point of care approach may be typically implemented with the novel technology. Optionally, once the BMA has been centrifuged, the resulting stratified cell layers may be prepared for delivery to the patient. Typically, the containers, anticoagulants used, and the delivery media used for interim storage and delivery are also prepared during this step. In some embodiments, preserving endogenous proteins, structure and morphology resides within this step, as too great a deviation from the oxygen, microenvironment and stress factors can lead to changes in the composition of the milieu. Typically, the proteins, structure, and morphology are not significantly altered. In one embodiment, the novel delivery media formulation is tailored to preserve the extracted cells and their endogenous factors, while maintaining cell health and identity. Typically, a premixture including an aqueous solution of anticoagulant (ACD-A), an equal amount of dextrose (50%), and phosphate buffered saline (PBS), or the like is pre-mixed and aliquoted in a volume to typically match or approximate the cellular matrix extracted from the centrifugation stratification layers at a ratio of about 1:1. More typically, the premixture is added to the cellular matrix with specific volumes being matched to, or slightly greater than 50/50 by volume, although the ratio may be greater, such as 2:1 or even higher. In some embodiments, the steps in extracting, isolating, separating, re-extracting, dosing, mixing, and delivery impact the cell population, endogenous proteins, surface structural and biomarkers, associated with the compositional regenerative capacity. Typically, the shorter the time consumed by the above-mentioned steps, the less adulterated the original milieu composition will become.

The skin-tissue treatment with MSCs disclosed herein may be used to repair, treat, or ameliorate various aesthetic or functional conditions (e.g., defects) of the skin tissue through augmentation. The MSCs of the present embodiments may provide an important resource for rebuilding or augmenting damaged or lost tissue. In addition, the MSCs may be used for augmenting soft tissue not associated with injury by adding bulk to a soft tissue area, opening, depression, or void in the absence of disease or trauma, such as for “smoothing”.

Naturally, further objects of the invention are disclosed throughout other areas of the specification, drawings, photographs, and claims.

It is understood that this invention is not limited to specific components, specific methods, specific implementation, or to particular compositions, and as such may, of course, vary. It is also understood that the terminology used herein is for the purpose of describing particular implementations only and is not intended to be limiting. Neither are mechanisms which have been provided to assist in understanding the disclosure meant to be limiting.

In one embodiment of the invention, disclosed is a method of treating deteriorated skin tissue in the face or selected areas of the body. The method comprises: (a) obtaining autogenous mesenchymal stem cells; (b) injecting said autogenous mesenchymal stem cells into the skin tissue; and, (c) improving and/or restoring the skin tissue towards a normal physiologic structure. The term normal physiologic structure is previously described in this description. In one aspect, the method further comprises obtaining autogenous bone marrow concentrate containing said autogenous mesenchymal stem cells. In another aspect, the method further comprises aspirating autogenous bone marrow to obtain said autogenous bone marrow concentrate. Illustratively, aspiration of said autogenous bone marrow results in obtention of about 5 milliliters to about 55 milliliters of said autogenous bone marrow concentrate. In another aspect, the method further comprises aspirating autogenous bone marrow of an ilium of a pelvis. In another aspect, treatment of deteriorated skin tissue with said autogenous mesenchymal stem cells comprises injecting said autogenous mesenchymal stem cells into said skin tissue. This injecting of said autogenous mesenchymal stem cells may be carried out at a depth of between about 0.5 millimeters to about 3.5 millimeters below the surface of the skin tissue. In any of the foregoing, injecting of said autogenous mesenchymal stem cells may comprise injecting an autogenous bone marrow concentrate containing said autogenous mesenchymal stem cells.

In another embodiment of the invention, disclosed is a method for treating deteriorated skin tissue that includes treatment of inflammation that is associated with the skin tissue deterioration. The method comprises: (a) obtaining autogenous mesenchymal stem cells; (b) injecting said autogenous mesenchymal stem cells into said skin tissue; (c) reducing the inflammation; and, (d) improving and/or restoring said skin tissue towards a normal physiologic structure. In one aspect, the method further comprises obtaining autogenous bone marrow concentrate containing said autogenous mesenchymal stem cells. The method further comprises aspirating autogenous bone marrow to obtain said autogenous bone marrow concentrate. Illustratively, the method further comprises aspirating said autogenous bone marrow to obtain about 5 milliliters to about 55 milliliters of said autogenous bone marrow concentrate. The method further comprises aspirating autogenous bone marrow of an ilium of a pelvis. The method further comprises minimally manipulating the autogenous bone marrow by processing it as described in Pettine, K. A., U.S. Pat. No. 9,408,874 (Aug. 9, 2016), which is incorporated herein by reference in its entirety. The method further comprises injecting said autogenous mesenchymal stem cells into said skin tissue. The method further comprises injecting said autogenous mesenchymal stem cells into said skin tissue at a location of between about, and at a depth of between about 0.5 millimeter to about 3.5 millimeters. The method of any of the foregoing further comprises injecting an autogenous bone marrow concentrate containing said autogenous mesenchymal stem cells. The method further comprises injecting autogenous bone marrow concentrate containing said autogenous mesenchymal stem cells into said skin tissue; which further comprises injecting between about 0.5 milliliters to about 3.5 milliliters of said autogenous bone marrow concentrate containing said autogenous mesenchymal stem cells into said skin tissue; which further comprises injecting between said about 0.5 milliliters to about 3.5 milliliters of said autogenous bone marrow concentrate containing said autogenous mesenchymal stem cells into said skin tissue at a rate of about 1 milliliter per about 1 seconds to about 5 seconds. In another aspect, the method in the foregoing comprises improving and/or restoring said skin tissue towards a normal physiologic structure.

BMA (bone marrow aspirate) and BMC (bone marrow concentrate) are known to contain hematopoietic as well as MSC populations. The method of extraction is correlative to the source of these cells. A novel technology utilizes an approach, whereby approximately 10 cc of BMA is drawn with frequent rotation and repositioning. Additional draws are done after deeper placement of a needle in the iliac crest.

Bone marrow collection and processing to BMC is carried out herein as described in Pettine, K. A., U.S. Pat. No. 9,408,874 (Aug. 9, 2016), which is incorporated herein by reference in its entirety. This provides a pre-determined amount of a processed bone marrow concentrate with a pre-determined amount of a pre-mixture, where the pre-mixture includes quantities of anticoagulant solution, dextrose and phosphate buffered saline. The iliac crest, and more the posterior iliac crest, is where bone marrow aspirate may be harvested in a surgical setting, however any suitable area where BMA may be extracted may be used. The novel cellular “snapshot’ is derived via timely methodologies specific to non-expanded, minimally manipulated, autologous cell and associated endogenous microenvironment (“milieu”). Once extracted, the BMA and the resulting BMC is obtained by isolation of the desired cell populations via centrifugation. However, any suitable means may be used to obtain the BMC from the BMA. When centrifugation is used, the BMA is separated according to the slightly differing specific gravities of the aspirated cell types. The cells contained in the BMA can be stratified under centrifugation. The volume, rate, and time of centrifugation are important for controlling the resulting biologic factors contained within the endogenous milieu. The longer the processing time and/or the more agitation and handling, the lower the oxygen level in the extracted cells which include cellular constituents and components sequestered in the residual endogenous milieu, wherein the milieu includes antigens, surface biomarkers, proteins and growth factors for angiogenesis, osteogenesis, other regenerative outcomes, and the like. In some embodiments, the degradative manipulation and resultant influence during processing is limited. The resulting, unadulterated milieu may retain a large number of unchanged biologic drivers, markers and signals that are dose appropriate and specific to the cascade of healing found through the native physiology. The stratification and selection of MSCs and progenitors from this population may influence traits such as specific plasticity and immunomodulation. The time between extraction and re-implantation or re-injection is within 1 hour, and may be within 30 minutes, or within 20 minutes. Thus, a point of care approach may be implemented with the novel technology. Optionally, once the BMA has been centrifuged, the resulting stratified cell layers may be prepared for delivery to the patient. The containers, anticoagulants used, and the delivery media used for interim storage and delivery are also prepared during this step. In some embodiments, preserving endogenous proteins, structure and morphology resides within this step, as too great a deviation from the oxygen, microenvironment and stress factors can lead to changes in the composition of the milieu. The proteins, structure, and morphology are not significantly altered. In one embodiment, the novel delivery media formulation is tailored to preserve the extracted cells and their endogenous factors, while maintaining cell health and identity. A premixture including an aqueous solution of anticoagulant (ACD-A), an equal amount of dextrose (50%), and phosphate buffered saline (PBS), or the like is pre-mixed and aliquoted in a volume to match or approximate the cellular matrix extracted from the centrifugation stratification layers at a ratio of about 1:1. The premixture may be added to the cellular matrix with specific volumes being matched to, or slightly greater than 50/50 by volume, although the ratio may be greater, such as 2:1 or even higher. In some embodiments, the steps in extracting, isolating, separating, re-extracting, dosing, mixing, and delivery impact the cell population, endogenous proteins, surface structural and biomarkers, associated with the compositional regenerative capacity. The shorter the time consumed by the above-mentioned steps, the less adulterated the original milieu composition will become.

In a further embodiment, the present invention is directed to methods of using the BMC or MSCs of the present invention for restoring deteriorated skin tissue, such as in age-related tissue loss in the face and selected areas of the body. In one embodiment, the present invention provides for a method for injecting the BMC or MSCs of the present invention into the dermis or hypodermis of a subject in need thereof. Injection of the BMC or MSCs of the invention into the subject may be carried out over one or more sessions within a treatment period (e.g., 1 to 10 weeks). The number of sessions may be from 1 to 10 (e.g., 1, 2, 3, 4, 5, 6, 7, 8, 9, and 10). The frequency of injection may range from 1 to 3 times per week. Preferably, three sessions of injections are administered sequentially. In some embodiments, the three sessions of injections are administered within a one week to three month time period (e.g., 1, 2, 3, 4, 5, 6, 7, 8, 9, 10 11, or 12 week time period), and preferably within a month of the first treatment.

In one embodiment, the treatment method comprises the steps of obtaining the BMC or MSCs of the invention autogenously, and injecting the BMC or MSCs into the dermis and/or the hypodermis at one or more areas of the face or selected areas of the body of a person, thereby restoring deteriorated skin tissue in the face or the selected areas of the body of the person.

The suitable areas, or injection sites, include, but are not limited to, the periorbital area, the lips, the malar area, the nasolabial folds, the labio-mandibular folds, and selected areas of the body, such as the neck, and the hands. The periorbital area includes the eyelids and surrounding areas including the eyebrows, bony eye socket and rims, cheeks and forehead. The malar area includes the cheek or the side of the head. The nasolabial folds are the deep folds which run from the side of the nose to the corner of the mouth. The labio-mandibular folds are the folds between the corner of the mouth and the jawbone.

The skin-tissue treatment with MSCs disclosed herein may be used to repair, treat, or ameliorate various aesthetic or functional conditions (e.g., defects) of the skin tissue through augmentation. The MSCs of the present embodiments may provide an important resource for rebuilding or augmenting damaged or lost tissue. In addition, the MSCs may be used for augmenting soft tissue not associated with injury by adding bulk to a soft tissue area, opening, depression, or void in the absence of disease or trauma, such as for “smoothing”.

The treatment can be performed without local or general anesthesia. A patient is placed in a treatment room, the BMC or MSCs are aspirated from the patient and prepared with minimal manipulation as described below, and the injections of the BMC or MSCs are given using a needle (e.g., a 30 gauge needle) with a syringe (e.g., a 3 to 5 cc syringe). In some embodiments, the BMC or MSCs are injected at various areas at the hypodermis level.

In a further embodiment, the method comprises making one or more injections of the BMC or MSCs. For example, in the case where two injections are made, a first injection into the dermis may be made at one or more areas of the face or selected areas of the body of a person, and a second injection into the hypodermis may be made at the same or different areas of the face or selected areas of the body, thereby resulting in improvement and/or restoration of the deteriorated tissue in the face or selected areas of the body of the person. Furthermore, the one or more injections may include the same quantity or different quantities of autogenous BMC or MSCs. For example, in the case where two injections are made, the first injection may include a certain quantity of the autogenous BMC or MSCs and the second injection may include a different quantity of the autogenous BMC or MSCs.

The one or more injections of BMC or MSCs may be performed without local or general anesthesia. For example, in this procedure, a needle (e.g., 30-gauge needle) with a syringe (e.g., a 3 to 5 cc syringe) can be used to inject the first quantity of BMC, SVF or MSCs at selected areas at the dermis level first. With, for example, 0.1 mL increments, the dermis of a specific area is treated. Upon completing the dermal injection, the 30-gauge needle length may be changed for injection in the subcutaneous tissue (hypodermis). Alternatively, the needle can be changed to a longer one for subcutaneous injection. The second injection is then made in the same selected areas, at the hypodermis level. Optionally, multiple sessions of injections are administered sequentially within the time periods disclosed herein.

Various types of injectors may be used with embodiments of the current invention.

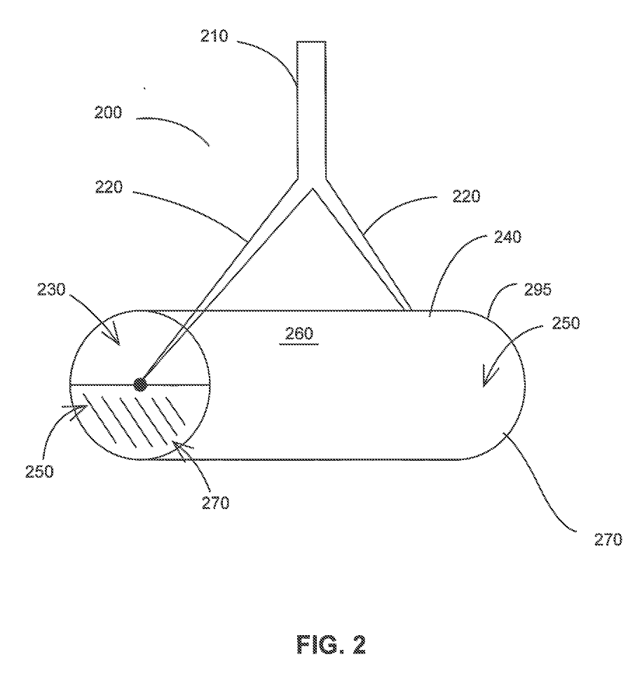

FIG. 2 is an illustration of a micro needling injector for injection of mesenchymal cells. The roller 200 comprises a handle 210 which connects to a pair of hollow tubes 220. It is understood that there may be a single hollow tube 220 or more than two hollow tubes 220. The number of hollow tubes 220 will match the number of roller units 240 that a particular injector 200 utilizes. The hollow tubes 220 connect, at an axle 280 of the roller, with an opening 290. The opening 290 allows the chamber, or reservoir, 230 to be filled with a fluid and pressurized to force the fluid out of the micro needles. A plate 250 in the chamber, or reservoir, separates the empty chamber, or reservoir, 230 from the stem cell chamber, or reservoir, 270. The plate 250 attaches to a hinge that rotates to separate stem cell chamber, or reservoir, 270 from the empty chamber, or reservoir, 230. The surface 260 of the roller units is penetrated with needles that accept stem cells from said chamber, or reservoir, 230. Stem cells are injectable into a patient via said needles.

In operation, the roller 200 may be rolled on the skin, on the face of a patient, for example. Each roller 240 has a particular length matrix of micro needle 295 on the surface of the roller unit 240. Each stem cell chamber, or reservoir, 270 is filled with a stem cell liquid described in this description of the invention. The stem cell chamber, or reservoir, 270 is filled through the handle 210 and the hollow tubes 220. The act of using the roller 200 may be through use of a syringe or bulb to squeeze or plunge air to force liquid through needles as the roller 200 is utilized. The plurality of micro needles 295 penetrate through the round surface 462 and allow passage of a fluid from the cell chamber, or reservoir, 270 through the plurality of needles. A matrix is an array of the micro needles. The matrix of micro needles may be arranged rectangularly having a plurality of rows and columns. The distance between the individual needles may be variable depending on an anticipated end use. For instance, a matrix of needles intended for use on one body part may be different than a matrix of needles intended for a different body part. It is understood for all variations of injectors comprising matrices of needles detailed below in the figures that a matrix of needles is on a surface of the injector as indicated. The matrix may penetrate the particular surface so that fluid from a reservoir may pass from the reservoir through the needles in the matrix and into the skin of a patient during an injection. The matrix may also be non-rectangular in that the arrangement of needles may not be in a row and column arrangement.

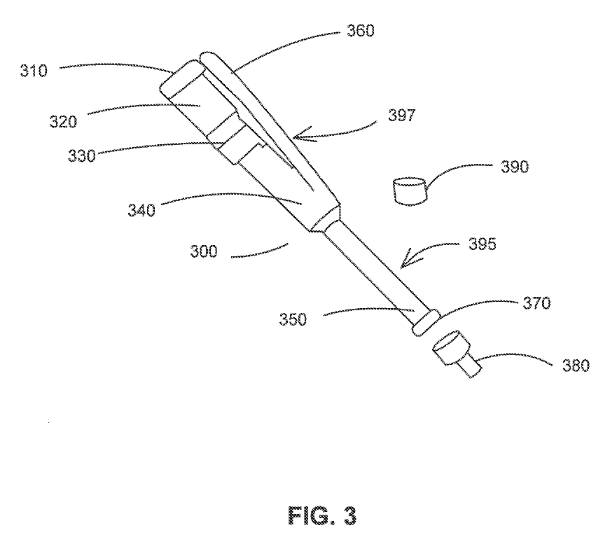

FIG. 3 is an illustration of a needle free liquid injector of mesenchymal cells. A needle free injector 300 may comprise an end cap 310, a gas cartridge 320, a safety 330, a plunger 340, a piston 350, an activation lever 360, an orifice 770, a luer cap, and a sterility cap.

The needle free injector 300 has a drug chamber, or reservoir, and provides for self-administration. The injector 300 may be composed of plastic elements. A chamber, or reservoir, 395 of the injector provides for passage of a drug and serves as a skin contacting surface. The chamber, or reservoir, 395 is comprised of the orifice 370 through which the drug enters the skin of a patient when the injection occurs. The diameter of the orifice may be approximately 100 um (micro meters). The chamber, or reservoir, 395 fires drug particles (in this case the mesenchymal cells) at a speed of approximately 100 meters per second, and which may reach a depth of 2 millimeters in the skin. An orifice of 0.127 millimeters may be used, which is comparable to a 25-gauge needle.

A pressure source 397 may comprise the plunger 340. The pressure source 397 may be a mechanical device which stores energy in a spring and releases the energy by pushing a plunger 340 to provide the necessary pressure. The pressure source may also be a pressure storage device that utilizes compressed gas in a gas cartridge 320. The compressed gas may be carbon dioxide or nitrogen, or other gas as those skilled in the art can understand. Pressurized metal air cartridges may be provided for access in portable units.

The injector 300 generates force by using compressed gas to propel a liquid (such as mesenchymal cells) through an orifice 370 at a high speed. An ultra-fine stream of fluid penetrates the skin layers which delivers the liquid into systemic circulation. The time required to deliver an injection may be about ⅓ of a second. High pressure generated in the injector 300 by the fluid in intimate contact with the skin allows the liquid to punch a hole into the skin and be delivered into the tissues in and under the skin. In a liquid injector 300 the orifice may be from 150 um to 300 um.

The impact of the piston 350 on a liquid chamber, or reservoir, 395 increases pressure and shoots a jet out of the nozzle has high velocity. The effect of the jet on the skin surface starts the formation of a hole in the skin through erosion, fracture, or other skin failure mechanism. Further impingement of the jet increases the depth of the hole in the skin. If the volumetric rate of the hole formation is less than the volumetric rate of jet impinging the skin, then some of the liquid may splash back toward the injector. The accumulation of liquid in the hole occurs because of a deeper hole in skin which slows down the incoming jet. Further development of a hole is stopped. The dimensions of the hole are established very early in the process from the time of impact of the jet. Stagnation of the jet at the end of the hole disperses the liquid into the skin in a near-spherical shape.

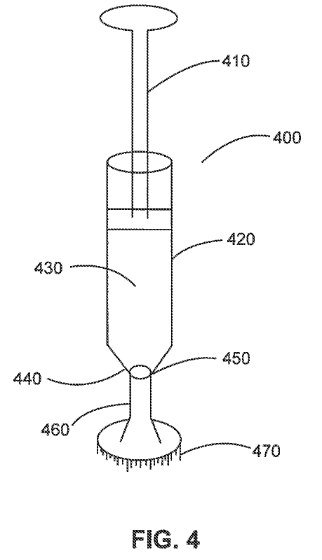

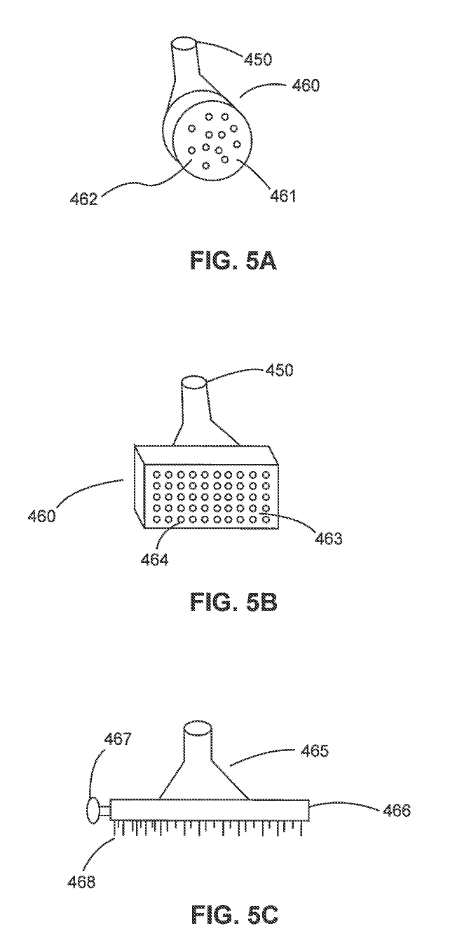

FIG. 4 is an illustration of a syringe liquid injector for injection of mesenchymal cells. A syringe liquid injector 400 may comprise a plunger 410, a standard syringe 420, a luer lock 440, an attachment 450 which may be an attachment to a standard syringe 420, a disposable needle injector, an a plurality of needles. A matrix of needles 470 is on the surface (See FIG. 5A element 461 on surface 462).

A sample of stem cells 430 may be placed in the syringe 420. In operation, the matrix of needles 470 are inserted into the skin of a patient to inject the stem cells 430 through the matrix of needles 470 into the dermis by pushing down on the plunger 410. The syringe may be filled with 10 cc's of stem cells. The matrix of needles 470 size may range from about 25 gauge to 30 gauge. The matrix of needles 470 may be of various length. For example, the needles may be 0.5 mm, 1 mm, 1.5 mm, 2.0 mm 2.5 mm, or 3.0 mm in length. The disposable needle injector may have any shape to fit, or accommodate, shapes of the face or other portion of the body.

FIG. 5A shows an embodiment of a round needle injector. The round needle injector 460 comprises a round surface 462. A plurality of needles 461 are in a matrix are on the round surface 462 and face away from the surface 462 so as to be injectable into a patient. A fluid may pass from a reservoir through the matrix of needles 461.

FIG. 5B shows an embodiment of a rectangular needle injector. The rectangular needle injector 460 comprises a rectangular needle surface 463. A plurality of needles 464 are in a matrix are on the rectangular surface 463 and face away from the surface 463 so as to be injectable into a patient. A fluid may pass from a reservoir through the matrix of needles 464.

FIG. 5C shows an embodiment of an adjustable needle injector 465. The adjustable needle injector 465 comprises an attachment to a plunger and syringe luer lock, a base 466, a plurality of needles 468 in a matrix and an adjuster 467 to change the needle depth from approximately 0.5 mm to 3.0 mm in a single attachment. Those skilled in the art will understand the workings of the adjustable needle length. A fluid may pass from a reservoir through the matrix of needles 468.

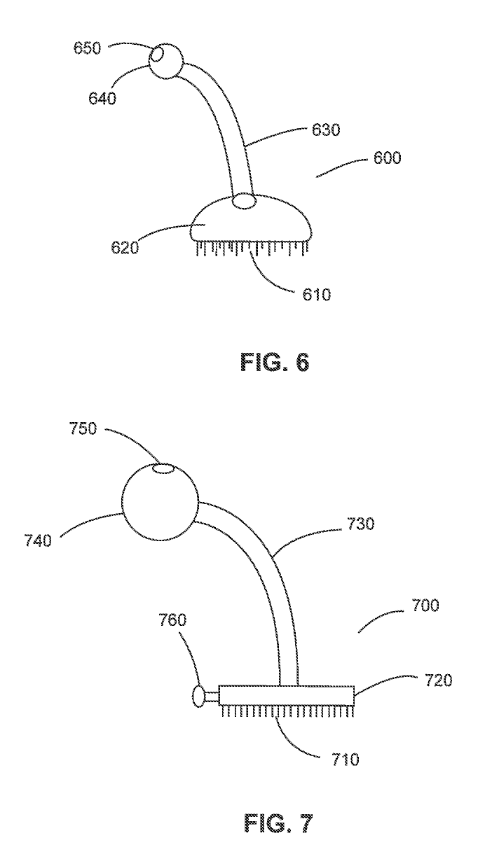

FIG. 6 shows an illustration of a pouch injector. The pouch injector 600 comprises a plurality of needles 610, a pouch, or reservoir, 620 fillable with stem cells, and a detachable flexible tubing 630, a squeeze filler 640 and an injection port 650. The pouch 620 is filled with stem cells, approximately 5 cc for example. The matrix of needles 610 are injected into the skin of a patient. The pouch 620 is squeezed to inject stem cells into the patient. The flexible tubing 630 may be attached to the pouch 620. The squeeze filler 640 is filled with stem cells. Squeezing the squeeze filler 640 pushes stem cells into the pouch 620. The flexible tubing may be from 6 cm to 10 cm thick. A fluid may pass from a reservoir through the matrix of needles 610.

FIG. 7 shows an illustration of a pouchless squeeze injector. The pouchless squeeze injector 700 comprises a plurality of needles 710 in a matrix, a base 720 that holds the needles, a flexible tubing, a squeeze injector 740 that may hold up to 5 cc of stem cells in a reservoir, and an injection port 750 that allows insertion of stem cells into the injector 700. An adjustor 760 may be used to change the needle depth from approximately 0.5 to 3.0 mm. The squeeze injector 740 is filled with stem cells. Squeezing the squeeze injector 740 causes stem cells to be pushed through the flexible tubing and out through the base 720 and matrix of needles 710. A fluid may pass from a reservoir through the matrix of needles 710.

FIGS. 2-4 and 6-7 show embodiments of injectors as described above. An injector is a device, apparatus or system capable of delivering a fluid. An injector is capable of pushing a fluid through a plurality of needles that may be in a matrix. For instance, an injector may comprise a plunger that is capable of applying a pressure to a liquid to force the liquid through a matrix of needles. An injector may be any device that is capable of applying pressure to cause a fluid to flow through a matrix of needles. An injector may further comprise a cylindrical roller comprising a hollow chamber or reservoir, a surface and a plate that separates said chamber into an empty chamber and a fluid chamber connected to at least one hollow tube capable of supplying fluid to the cylinder or reservoir, and wherein the needle matrix penetrate the surface to accept fluid from the fluid chamber or reservoir. An injector may also further comprise a syringe with a reservoir and which is able to attach to a shaped disposable needle matrix having a flat surface, and wherein the needle matrix penetrates the surface to accept fluid from the fluid chamber or reservoir. An injector may also further comprise a pouch reservoir with a surface having a matrix of needles. The pouch is a reservoir that is able to force a liquid through the matrix of needles.

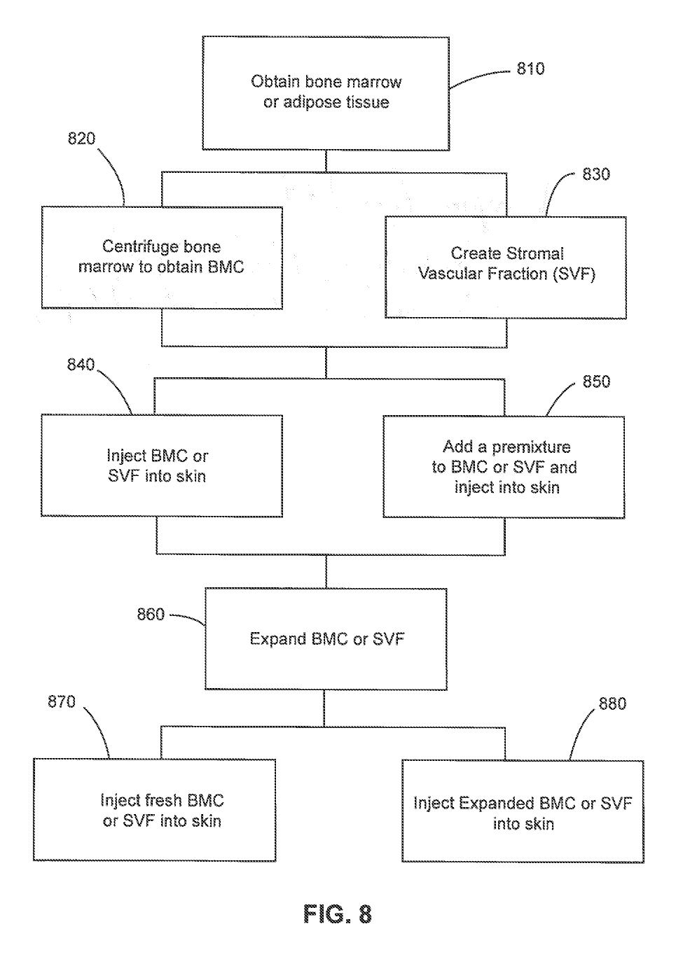

FIG. 8 is a flow diagram of an illustration of a method of an embodiment of the invention. The embodiment of FIG. 8 shows method of the invention comprises obtaining a bone marrow or adipose tissue sample 810. The sample may be autogenous or allogeneic. If a sample is obtained from bone marrow, the bone marrow is then centrifuged to obtain a bone marrow concentrate (BMC) 820. If the sample is obtained from adipose, a stromal vascular fraction (SVF) is created from the adipose tissue 830. The BMC or the SVF is then injected into the skin 840. Also, the BMC or SVF may be added to a pre-mixture as described herein and then injected into skin 850. Cells obtained from steps 820 or 830 may then be expanded 860. The process of cell expansion is within the skills of those skilled in the art. Another injection of fresh BMC or SVF cells 870 or an injection of expanded BMC or SVF cells may occur 880. Steps 870 and 880 may be repeated as often as possible and desirable.

Embodiments of the invention may provide a combination of a pre-mixture of Bone Marrow concentrate or mesenchymal stem cells with an amount of platelet rich plasma (PRP). PRP may provide additional tissue repair and regeneration effects on skin tissue and elsewhere. PRP may be used in a wide range of applications in the cosmetic and dermatological fields and as a composition suitable for dermal injection comprising PRP and mesenchymal stem cells or BMC for regenerating action for relaxing expression wrinkles, a lifting effect for sustaining relaxed skin tissues, for scar reduction and wound healing. The composition of PRP and BMC or mesenchymal stem cells performs a regenerating and a repairing action on the dermal skin cell layers, increasing vitality and longevity of the skin, scalp and hair follicles. The PRP releases numerous growth factors to stimulate the biology of the mesenchymal stem cells and fibroblast. The combination of PRP and mesenchymal stem cells or BMC is formulated to provide the vital function of platelet growth factors while combining a gradual, more physiological releasing rate, and stimulating the recruiting of stem cells all to stimulate the production and function of fibroblasts.

The use of PRP for skin regeneration in combination with mesenchymal stem cells or BMC may include a PRP concentration of at least 900,000 platelets. The PRP is added to a pre-mixture of BMC containing mesenchymal stem cells for the purpose of producing and stimulating fibroblasts. The PRP utilized may be autologous platelets. The PRP utilized may be allogenic platelets.

Generally, plasma is a fluid tissue. Plasma contains molecules and cells that may regulate key processes involved in tissue repair, including proliferation, chemotaxis, migration, cellular differentiation and extracellular matrix synthesis and remodeling. PRP is blood plasma that has been enriched with platelets. Blood is made of red blood cells, white blood cells, plasma, and platelets. Platelets, also known to be responsible for blood clotting, release a multitude of growth factors including Platelet-derived growth factor (PDGF), a potent chemotactic agent, and TGF beta, which stimulates the deposition of extracellular matrix. Both of these growth factors have been shown to play a significant role in the repair and regeneration of connective tissues. Within PRP, the increased number of platelets delivers an increased number of growth factors to the topical area.

There are seven principal known growth factors in PRP: Platelet Delivered Growth Factors as (PDGFaa), PDGFab), Transforming Growth Factor beta-, (TGF-b), TGF-b2, Vascular Endothelial Growth Factor (VEGF) and Epithelial Growth Factor (EGF). As a concentrated source of platelets, PRP contains (and releases through degranulation) several different growth factors (cytokines) that stimulate tissue regeneration. PRP may also be used to treat sports injuries in athletes.

Platelets are non-nuclear cellular fragments derived from megakaryocytes in the bone marrow through controlled cellular fragmentation. Platelets are specialized secretory cells that release the contents of their intracellular granules in response to activation. Platelets contain a complete array of pre-synthesized protein molecules, among which are the high presence of cytoskeletal proteins, signaling proteins, membrane proteins, protein-processing proteins, and cytoskeleton regulatory proteins.

Platelet secretory granules contain growth factors (GFs), coagulation proteins, cell-activating molecules, cytokines, integrins, which are synthesized in megakaryocytes and packaged into the granules through vesicle trafficking processes. Three major storage compartments in platelets are alpha granules, dense granules, and lysosomes. The majority of the substances are contained in alpha granules. Due to the large stores of cytokines and growth factors that are normally released during clot formation at wound sites, platelets, and consequently PRP, release cytokines and growth factors in the neighboring milieu.

The PRP may be a PRP where the platelets are autologous and therefore the donor and the recipient are the same person. The autologous PRP is thereby derived from the exact biological background, and therefore the PEP derived growth factors are in a favorable situation for inducing skin regeneration. Furthermore, being that PRP is an organic fluid belonging to the donor, a total safe use is also guaranteed.

The PRP may also be PRP wherein the platelets are allogenic. These platelets may be allogenic single donor platelets units from the blood bank that were ABO and RhD matched, virus checked, leukocyte depleted, irradiated and activated by human thrombin.

The number of platelets in the blood is referred to as the platelet count and is normally between 150,000 to 450,000 per microliter (ul) of blood. The platelet growth factors content is variable among individuals and it is not necessarily proportional to the platelet count.

The PRP, processed as noted above and containing platelets, is mixed with BMC for placement into the dermis for skin regeneration.

Together with the methodologies and systems described above, a method and system of preparation of PRP for use in combination with BMC containing mesenchymal stem cells may comprise collecting blood in a test tube, separating the platelets by centrifugation, and mixing the platelets obtained with calcium chloride until the concentration of at least 900,000 platelets/ul is obtained. After centrifuging the plasma to produce concentrated platelets contained in a sterile canister, the PRP is carefully combined with a premixture of BMC also in a sterile canister. The PRP is slowly added to the BMC with a syringe to minimize any cell lysis.

The cosmetic composition of PRP and BMC containing mesenchymal stem cells may contain a PRP concentration from about 25% to 50% of the total volume of the BMC/PRP pre-mixture. The exact ratio of PRP to BMC containing mesenchymal stem cells may vary based on the age, skin condition and decision of the practitioner.

As discussed, PRP therapy and treatment embodiments of this invention involves centrifuging a person's blood until it contains a concentrated mix of plasma cells and growth factors and then injecting the resulting substance in combination with BMC containing mesenchymal stem cells directly into the dermis. The PRP release of growth factors directly stimulates the biologic function of fibroblasts located in the dermis. The PRP also stimulates the biology of the mesenchymal stem cells to replicate itself and also differentiate into creating more fibroblasts.

Platelets are sensitive to process induced stress, from blood extraction to PRP composition production. Thus, the amount of platelet-derived factors available at the end of the manipulation process depends on cumulative effects over platelets, starting from phlebotomy and ending with formation of a PRP composition. The platelet concentration of at least 900,000 platelets/ul, corresponds to two to six fold platelet concentration in whole blood. In some embodiments of the invention, platelet concentrations of 1,000,000 platelets/ul to 1,500,000 platelets/ul may be used. Further, the PRP concentration of a composition may be from 0.1% to 5.0% of the total weight of the composition.

A composition according to embodiments of the invention combine the benefits of skin regeneration utilizing both PRP and mesenchymal stem cells to produce more fibroblasts and stimulate fibroblasts.

The foregoing description is considered as illustrative of the principles of the invention. Furthermore, since numerous modifications and changes will readily occur to those skilled in the art, it is not desired to limit the invention to the exact construction and process shown and described above. Accordingly, resort may be made to all suitable modifications and equivalents that fall within the scope of the invention. The words “comprise,” “comprises,” “comprising,” “include,” “including,” and “includes” when used in this specification are intended to specify the presence of stated features, integers, components, or steps, but they do not preclude the presence or addition of one or more other features, integers, components, steps, or groups thereof.

### EXAMPLES

The following examples further illustrate specific embodiments of the invention. However, the following examples should not be interpreted in any way to limit the invention. Thus, it is understood that modifications which do not substantially affect the activity of the various embodiments of this invention are also provided within the definition of the invention provided herein. Accordingly, the following examples are intended to illustrate but not limit the present invention.

### Example 1

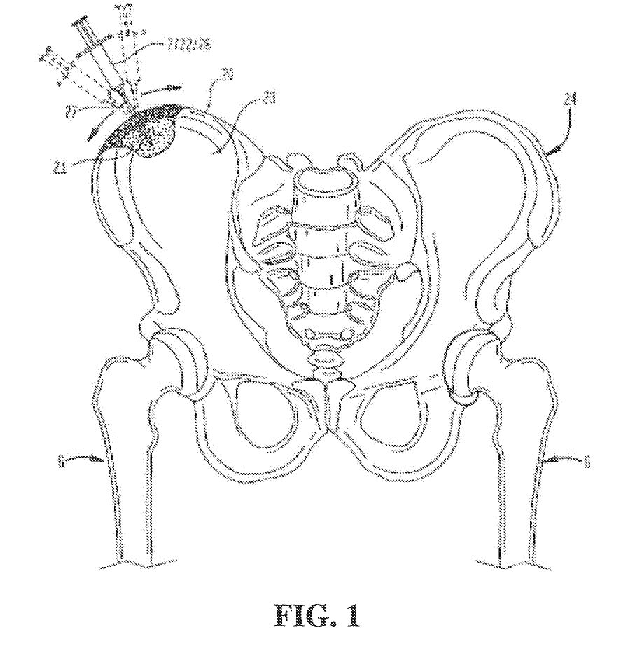

Obtention of Autogenous MSCs: Referring to FIG. 1, autogenous MSCs (2) are multipotent stromal cells that can differentiate into a variety of cell types, including: osteoblasts (bone cells), chondrocytes (cartilage cells), myocytes (muscle cells) and adipocytes (fat cells). Autogenous MSCs (2), suitable for use in embodiments of the inventive method, are obtained from bone marrow (21) or fractions thereof; however, this is not intended to limit obtention of autogenous MSCs (2) suitable for use with the inventive method solely from bone marrow (21) and it is understood that autogenous MSCs (2) may be isolated from other tissues such as: peripheral blood, synovium, periosteum, skeletal muscle, or adipose tissue. Additionally, as to certain embodiments, it is understood that autogenous MSCs (2), whether from bone marrow (21) (or fractions thereof) or other tissue sources, can optionally be expanded in media to obtain an amount of autogenous MSCs (2) for use in embodiments of the inventive method.

Bone marrow aspirates may have a cellularity of marrow (21) of about 15 to about 30 million mononuclear cells per milliliter (“MNC/mL”), whereas less than 10 million MNC/mL usually means a rather diluted sample provided the donor was healthy and the marrow not fibrotic. The average colony forming unit-fibroblasts (“CFU-f”) content of healthy human bone marrow is about 100 CFU-f/million MNC. Accordingly, bone marrow concentrate (22) (“BMC”) resulting from aspirating bone marrow (21) can contain a few thousands (about 1500 to about 3000) CFU-f/mL of bone marrow (21).

As to some embodiments, 3 to 4 separate aspirations may be pooled (around 15-20 mL total) of bone marrow (21). The pooled aspirates of the bone marrow (21) can be diluted in cell culture medium (using platelet lysate instead of fetal bovine serum) and seeded directly into culture vessels without any further purging or manipulation. Non-adherent cells can be removed between 48 to 72 hours. First passage (P1) autogenous MSCs (20) meet the MSC criteria of the International Society for Cellular Therapy. This protocol can yield several hundred millions of autogenous MSCs (2) from the original aspirated bone marrow (21) within a few weeks.

As to some embodiments, autogenous MSCs (2) obtained from bone marrow (21) may be utilized in embodiments of the inventive method as autogenous BMC (22) without further processing; although autogenous MSCs (2) can be contained or entrained in other injectable or otherwise administrable biocompatible materials or compositions such as biocompatible hydrogels, or the like. One source of autogenous BMC (22) can be the bone marrow (21) of the ilium (23) of the greater pelvis (24); although autogenous BMC (22) obtained from other sites may be utilized.

### Example 2

Bone Marrow Collection and Processing: Following is an illustrative example of bone marrow collection and processing to BMC, which was carried out as described in Pettine, K. A., U.S. Pat. No. 9,408,874 (Aug. 9, 2016), which is incorporated herein by reference in its entirety for all that it discloses and teaches. Accordingly, 60 cc of bone marrow aspirate was collected over ACD-A as needed per process. The marrow was processed using the bone marrow concentration system according to the detailed protocol. The patient was given IV antibiotics and placed prone on an image table. Intravenous Versed and Fentanyl was administered and the skin was anesthetized with buffered 1% Lidocaine. The aspirator was rinsed and the syringes were transferred with heparin solution, approximately 1000 U/ml. The heparin solution coated the inner surface of the 60 cc aspiration needle and trephine needle. The remaining heparin was expelled from the syringe. 6 cc of ACD-A was aspirated into the 60 cc syringe. Bone marrow was aspirated from the posterior iliac crest when the patient was positioned prone on a fluoro table. The right iliac wing was prepped and draped according to standard surgical protocols. A trephine needle and 60 cc syringe was used to remove the marrow. The surgeon inserted the trephine needle percutaneously through the skin until the bony surface of the iliac crest was felt. Using a mallet, the needle was then inserted to a depth of 3-4 cm into the crest. This was accomplished with fluoroscopic guidance. A 60 cc syringe containing 6 ml of acid citrate dextrose anticoagulant solution (ACD-A) (10% of the final volume) was attached to the needle. The marrow was aspirated by pulling the plunger back and allowing the syringe to fill to the 10 cc level. The needle was repositioned by advancing 1.5-2 cm and an additional 10 cc of aspirate was obtained. This process was repeated until 60 cc of iliac aspirate was obtained. Once the final marrow volume was reached, the solution was mixed by gentle rocking of the syringe as the syringe was rotated on its long axis. The marrow was then ready for processing. The marrow was mixed with anticoagulant solution by gently turning the syringe after each 10 cc of aspirate collection. The extracted marrow was placed in an isolating canister and loaded into the centrifuge. The marrow was centrifuged for about 12 minutes at about 3200 rpm. The processed marrow was drawn with a syringe from the centrifuge and then rocked while rotating the syringe on its long axis. The syringe was then presented to a sterile field. The amount of bone marrow concentrate removed from the centrifuge equaled the amount to be injected. The cell delivery media was pre-mixed and aliquoted to 1 cc, composing of 0.5 cc of ACD-A and 0.5 cc of dextrose (50%). The delivery media was injected into a closed vial containing the cell components slowly, in order to homogenize the mixture and incorporate oxygen and turbidity mixing in a closed, sterile system.

Following is a second illustrative example of bone marrow collection and processing to BMC. Referring to FIG. 1, about 55 milliliters (“mL”) of autogenous bone marrow aspirate (26) (“BMA”) was collected over about 5 mL acid citrate dextrose-anticoagulant (“ACD-A”) from the participant's posterior iliac crest (20) of the ileum (23) of the greater pelvis (24). The procedure was performed with intravenous sedation consisting of VERSED® (also known as Midazolam) (8-chloro-6-(2-fluorophenyl)-1-methyl-4H-imidazo[1,5-a]-[1,4]benzodiazepine, CAS Number: 59467-70-8) and FENTANYL® (N-(1-(2-phenylethyl)-4-piperidinyl)-N-phenylpropanamide, CAS Number 437-38-7). Positioning of a JAMSHIDI® bone marrow biopsy needle (27) in the iliac crest (20) was confirmed by fluoroscopy. BMA (26) was collected in a 60 mL syringe in a series of discrete pulls on the plunger (targeting a collection of 5-10 mL per pull), with repositioning of the needle tip between pulls based on the reported enrichment of autogenous MSCs (20) as described by Hernigou, et al., J Bone Joint Surg Br 2005 87 (7) 896-902. The BMA (26) was captured using a ART21® BMC cell capturing device available from Celling Biosciences, Austin, Tex. to obtain BMC (22). A BMC (22) volume of about 12 mL was drawn from the ART21® BMC cell capturing device.

### Example 3

Protocol for Treatment of Skin Tissue. The following is a technique used to treat deteriorated skin tissue, resulting in improvement and/or restoration of the skin tissue. The technique involves the following. Any biologic mesenchymal stem cell product can be utilized

Obtention of Autogenous MSCs: Referring to FIG. 1, autogenous MSCs (2) are multipotent stromal cells that can differentiate into a variety of cell types, including: osteoblasts (bone cells), chondrocytes (cartilage cells), myocytes (muscle cells) and adipocytes (fat cells). Autogenous MSCs (2), suitable for use in embodiments of the inventive method, are typically obtained from bone marrow (21) or fractions thereof; however, this is not intended to limit obtention of autogenous MSCs (2) suitable for use with the inventive method solely from bone marrow (21) and it is understood that autogenous MSCs (2) may be isolated from other tissues such as: peripheral blood, synovium, periosteum, skeletal muscle, or adipose tissue. Additionally, as to certain embodiments, it is understood that autogenous MSCs (2), whether from bone marrow (21) (or fractions thereof) or other tissue sources, can optionally be expanded in media to obtain an amount of autogenous MSCs (2) for use in embodiments of the inventive method.

Typically, bone marrow aspirates can have a cellularity of marrow (21) of about 15 to about 30 million mononuclear cells per milliliter (“MNC/mL”), whereas less than 10 million MNC/mL usually means a rather diluted sample provided the donor was healthy and the marrow not fibrotic. The average colony forming unit-fibroblasts (“CFU-f”) content of healthy human bone marrow is about 100 CFU-f/million MNC. Accordingly, bone marrow concentrate (22) (“BMC”) resulting from aspirating bone marrow (21) can contain a few thousands (about 1500 to about 3000) CFU-f/mL of bone marrow (21).

As to particular embodiments, autogenous MSCs (2) obtained from bone marrow (21) or adipose tissue may be utilized in embodiments of the inventive method as autogenous BMC (22) or adipose-derived stromal vascular fraction (SVF) without further processing; although autogenous MSCs (2) can be contained or entrained in other biocompatible materials or compositions known in the relevant art. One preferred source of autogenous BMC (22) can be the bone marrow (21) of the ilium (23) of the greater pelvis (24); although autogenous BMC (22) obtained from other sites may be utilized. Adipose tissue is typically suctioned from the abdomen but other anatomical locations can also be used. Adipose-derived SVF, as has been described in the art, may be used; for example, as described by Oberbauer, E., et al., “Enzymatic and Non-Enzymatic Isolation Systems for Adipose Tissue-Derived Cells: Current State of the Art,” Cell Regeneration, 215, 4:7, the disclosure of which is incorporated herein by reference in its entirety.

### Example 4

Expanding Human MSCs (hMSCs). An embodiment of the disclosed invention provides MSC preparations and/or formulations, and associated methods, for treating skin tissue using autogenous or allogeneic MSCs, wherein the MSCs have been cultured and/or culture-expanded. Evidence exists that this may enhance the efficacy as well as safety of hMSC therapeutics. Various techniques and methods for expanding the MSCs are well known and have proliferated in the art, including all aspects of preparing the culture media, cell bioprocessing protocols, and related steps, while maintaining the therapeutic and differentiation capacity of the MSCs. For the purpose of this invention, the following publications encompass the techniques and methods for expanding the MSCs of the invention prior to their use in treatment of skin tissue. Thus, the disclosures of the following publications are hereby incorporated by reference in their entirety for all that they disclose and teach:
Jung, S., et al., “Ex Vivo Expansion of Human Mesenchymal Stem Cells in Defined Serum-Free Media,” Stem Cells International, Volume 2012, Article ID 123030, pages 1-21.
Bruedigam, C., et al., “Basic Techniques in Human Mesenchymal Stem Cell Cultures: Diferentiation into Osteogenic and Adipogenic Lineages, Genetic Perturbations, and Phenotypic Analyses,” Curr. Protoc. Stem Cell Biol. 17:1H.3.1-1H.3.20.
Battula, V. L., et al., “Human placenta and bone marrow derived MSC cultured in serum-free, b-FGF-containing medium express cell surface frizzled-9 and SSEA-4 and give rise to multilineage differentiation,” Differentiation (2007) 75:279-291.
Ikebe, C., et al., “Mesenchymal Stem Cells for Regenerative Therapy: Optimization of Cell Preparation Protocols” BioMed Research International, Volume 2014, Article ID 951512, pages 1-11.

### Example 5

Obtaining Allogeneic-Derived MSCs. An embodiment of the disclosed invention provides MSC preparations and/or formulations, and associated methods, for treating skin tissue using either autogenous or allogeneic MSCs. Regarding allogeneic MSCs, methods and sources of obtention have proliferated in the art in recent years. Illustratively, the allogeneic MSCs may be obtained from the iliac wing, the removed bone from a total hip or knee replacement, or from other appropriate aspiration sites from one or more screened donors. For the purpose of this invention, the foregoing publications in Example 4 above by Jung et al., Bruedigam et al., Battula et al., and Ikebe et al. encompass the techniques and methods for obtaining the allogeneic MSCs of the invention; and, accordingly, the disclosures of these four publications are hereby incorporated by reference in their entirety for that purpose. Additionally, various companies have emerged that provide specialized culture media to produce MSCs that have the ability to maximize fibroblast differentiation and increase collagen fiber production, elastin fiber production, and glycosaminoglycan production, as well as to maximize production of various growth factors, including bFGF, KGF-2, IGF-1, EGF, and SOD-1. For the purpose of this invention, the following is an illustrative list of companies that provide specialized culture media to produce the allogeneic MSCs of the invention: PromoCell, Life Cell Technology, StemPro®, StemMACS™, and Cell Applications Inc.

### Example 7

Inclusion of Additives to Induce Keratinocyte Proliferation and Collagen Production. An embodiment of the disclosed invention provides MSC preparations and/or formulations, and associated methods, for treating skin tissue, wherein the MSC culture media are supplemented with additives known to induce keratinocyte proliferation and collagen production. This is believed to be important because keratinocyte proliferation and collagen production are known to decrease with aging. Methods for inclusion of various additives that induce keratinocyte proliferation and collagen production have proliferated in the art in recent years. For the purpose of this invention, the foregoing publications in Examples 4-5 above by Jung et al., Bruedigam et al., Battula et al., and Ikebe et al. encompass additives, techniques and methods for inducing keratinocyte proliferation and collagen production; and, accordingly, the disclosures of these four publications are hereby incorporated by reference in their entirety for that purpose.

### Example 8

Post-Treatment of MSC-Treated Skin Tissue. An embodiment of the disclosed invention provides MSC preparations and/or formulations, and associated methods, for treating skin tissue, wherein after treatment of the skin tissue, one or more post-treatments of the skin tissue are undertaken to enhance the MSC function after application. It is well known in the art that optimization of penetration and/or delivery through skin tissue can significantly improve the overall outcome of the treatment, and a wealth of agents, methods and techniques have been developed for that purpose. Post-treatments have been reported to be effective in accomplishing as much as a 10-fold enhancement relative to procedures that do not employ post-treatment methods. Optimization of penetration and/or delivery can be accomplished, illustratively, by the use of plastic covers and/or masks, and the like; by modifications to the stratum corneum, e.g., by use of hydration agents and chemical enhancers; by ozonation; by ablation and follicular delivery; and/or by electrically assisted methods (e.g., ultrasound, iontophoresis, electroporation, magnetophoresis, laser light and photomechanical waves). Better optimization can be accomplished by synergistic use of a multiplicity of the foregoing methods. For the purpose of this invention, as contemplated herein, the following publications encompass the techniques and methods for post-treatment of MSC-treated skin tissue. Thus, the disclosures of the following publications are hereby incorporated by reference in their entirety for all that they disclose and teach:
Barry, B. W., “Novel Mechanisms and Devices to Enable Successful Transdermal Drug Delivery,” European Journal of Pharmaceutical Sciences, 14, (2001), 101-114.
Santus, G. C., et al., “Transdermal Enhancer Patent Literature,” Journal of Controlled Release, 25, (1993), 1-20.
Lane, M. E., “Skin Penetration Enhancers,” International Journal of Pharmaceutics, 447 (2013), 12-21.
Williams, A. C., et al., “Penetration Enhancers,” Advanced Drug Delivery Reviews, 64, (2012), 128-137.
Zhai, H., et al., “Occlusion vs. Skin Barrier Function,” Skin Research and Technology, 8, (2002), 1-6.

As can be easily understood from the foregoing, the basic concepts of the present invention may be embodied in a variety of ways. Embodiments of the invention involve numerous and varied ways of a treatment of skin tissue with autogenous and/or allogeneic mesenchymal stem cells resulting in improvement, restoration, rejuvenation, augmentation, and/or repair of said skin tissue towards normal physiologic structure.

It is to be understood that, as contemplated herein, in any and all instances throughout this application where Applicants refer to injection(s) using BMC, SVF or MSC, any and all of the various BMC, SVF or MSC delivery media or mixtures described herein may be used, whether the specific details of the BMC and/or MSC delivery media or mixture are recited or not.

As can be easily understood from the foregoing, the basic concepts of the present invention may be embodied in a variety of ways. The invention involves numerous and varied embodiments of a treatment of deteriorated skin tissue with autogenous mesenchymal stem cells resulting in improvement and/or restoration of said skin tissue towards normal physiologic structure.

As such, the particular embodiments or elements of the invention disclosed by the description or shown in the figures or tables accompanying this application are intended to be exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “treatment” should be understood to encompass disclosure of the act of a “treating”, whether explicitly discussed or not, and, conversely, were there effectively disclosure of the act of “treating”, such a disclosure should be understood to encompass disclosure of a “treatment” and even a “means for treating.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition each definition hereby incorporated by reference.

All numeric values herein are assumed to be modified by the term “about”, whether or not explicitly indicated. For the purposes of the present invention ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes, for example, the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

It is to be understood that, as used herein, the grammatical conjunction “and/or” refers throughout to either or both of the stated possibilities.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) a treatment of deteriorated skin tissue with autogenous mesenchymal stem cells herein disclosed and described, ii) the related methods disclosed and described, iii) similar equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

While the disclosure has been illustrated and described in detail in the figures and foregoing description, the same is to be considered as illustrative and not restrictive in character, it being understood that only selected embodiments have been shown and described and that all changes, modifications and equivalents that come within the spirit of the disclosures described heretofore and/or defined by the following claims are desired to be protected. It will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. In addition, all publications cited herein are indicative of the level of skill in the art and are hereby incorporated by reference in their entirety as if each had been individually incorporated by reference and fully set forth

What is claimed is:

1. A treatment method for improving or restoring deteriorated skin tissue in the face or selected areas of the body of a person, comprising:
obtaining mesenchymal stem cells from the person; and
using a needle matrix to inject said mesenchymal stem cells into said skin tissue, said mesenchymal stem cells being one of autogenous mesenchymal stem cells and allogeneic stem cells;
wherein said mesenchymal stem cells improve or restore said skin tissue.

2. The method of claim 1 further comprising:
obtaining bone marrow concentrate containing said mesenchymal stem cells.

3. The method of claim 2 further comprising:
aspirating said bone marrow to obtain said bone marrow concentrate.

4. The method of claim 3 further comprising:
aspirating said bone marrow from an ilium of a pelvis of said person.

5. The method of claim 3 wherein said bone marrow concentrate is minimally manipulated.

6. The method of claim 1 further comprising:
obtaining a stromal vascular fraction (SVF) from adipose.

7. The method of claim 1 further comprising:
making said one or more injections of said mesenchymal stem cells into the dermis or the hypodermis of said skin tissue.

8. The method of claim 1 further comprising:
making said one or more injections of said mesenchymal stem cells within less than one hour of obtention from said person.

9. The method of claim 7 wherein said step of injecting said mesenchymal stem cells comprises:
injecting deteriorated skin tissue in the face in the peri-orbital area, the lips, the malar area, the nasolabial folds, forehead or the labio-mandibular folds.

10. The method of claim 1 wherein said step of injecting said mesenchymal stem cells comprises:
injecting deteriorated skin tissue in the selected areas of the body in the neck, the hands or a traumatized or burned area.

11. A method for treating deteriorated skin tissue in the face or selected areas of the body of a person with mesenchymal stem cells, comprising:
aspirating autogenous or allogeneic bone marrow to obtain bone marrow aspirate containing said mesenchymal stem cells;
concentrating said bone marrow aspirate to obtain bone marrow concentrate, said bone marrow concentrate containing said mesenchymal stem cells; and
using a needle matrix to inject said bone marrow concentrate into said deteriorated skin tissue;
wherein said mesenchymal stem cells improve or restore said skin tissue.

12. The method of claim 11 further comprising:
aspirating said bone marrow from an ilium of the pelvis of a person.

13. The method of claim 11 wherein said step of aspirating autogenous or allogenic bone marrow comprises minimally manipulating said bone marrow.

14. The method of claim 11 wherein concentrating said bone marrow aspirate to obtain said bone marrow concentrate is done via centrifugation.

15. The method of claim 11 further comprising:
adding a premixture that includes an aqueous solution of an anticoagulant, an equal amount of dextrose, and an amount of phosphate buffered saline to said bone marrow concentrate to provide a delivery media formulation.

16. The method of claim 15 wherein said step of adding said premixture comprises adding a volume of said premixture substantially the volume of said bone marrow concentrate.

17. The method of claim 11 further comprising:
making said one or more injections of said bone marrow concentrate into the dermis or the hypodermis of said skin tissue.

18. The method of claim 11 further comprising:
making said one or more injections of said bone marrow concentrate or said adipose concentrate within less than one hour of obtention from said person.

19. The method of claim 18 wherein said one or more injections of said bone marrow concentrate or said adipose concentrate are made within less than 30 minutes of obtention from said person.

20. The method of claim 11 wherein the deteriorated skin tissue in the face is in the peri-orbital area, the lips, the malar area, the nasolabial folds, forehead or the labio-mandibular folds.

21. The method of claim 11 wherein the deteriorated skin tissue in the selected areas of the body is in the neck, the hands or traumatized or burned areas.

22. A method of treatment of skin tissue, said method comprising:
obtaining mesenchymal stem cells from a person; and
injecting said mesenchymal stem cells into said skin tissue with an injector using a matrix needle injector.

23. The method of claim 22 wherein said step of injecting said mesenchymal stem cells comprises:
using a micro-roller comprising a handle, a hollow tube connected to said handle, and a roller having a liquid chamber and a plurality of needles around said roller.

24. The method of claim 21, wherein said step of injecting said mesenchymal stem cells comprises:
using a syringe injector comprising a plunger, a luer lock and a disposable needle applicator.

25. The method of claim 22, wherein said step of injecting said mesenchymal stem cells comprises:
using a pouch injector comprising a flexible pouch fillable with stem cells and a plurality of micro-needles.

26. A method of treatment of skin tissue, said method comprising:
obtaining autogenous or allogeneic mesenchymal stem cells; and
injecting said mesenchymal stem cells into said skin tissue using a needle free injector.

27. An apparatus for treating skin tissue, said apparatus comprising:
a reservoir that supplies a fluid;
a micro needle matrix connected to said reservoir; and
an injector that pushes said fluid through said micro needle matrix and into said skin tissue.

28. The apparatus of claim 27 wherein said injector comprises a cylindrical roller with said reservoir inside said cylindrical roller, a surface and a plate that separates said reservoir into an empty reservoir and a fluid reservoir and wherein said cylindrical roller is connected to at least one hollow tube capable of supplying fluid to said reservoir, and wherein said micro needle matrix penetrate said surface to accept fluid from said fluid chamber.

29. The apparatus of claim 27 wherein said injector comprises a syringe liquid injector comprising shaped disposable needle injector with a flat surface capable of holding said matrix of needles and attachable to a syringe, and wherein said micro needle matrix penetrate said surface to accept fluid from said syringe.

30. The apparatus of claim 27, and wherein said injector comprises a pouch capable of holding a plurality of needles on a flat surface and said reservoir is inside said pouch and is fillable with a fluid, and wherein said micro needle matrix penetrate said surface to accept fluid from said syringe.

31. The apparatus of claim 27, wherein said injector comprises a means for adjusting a depth of penetration of said needles.

32. A method for treating deteriorated skin tissue in the face or selected areas of the body of a person with mesenchymal stem cells, comprising:
aspirating bone marrow to obtain bone marrow aspirate containing said mesenchymal stem cells;
concentrating said bone marrow aspirate to obtain bone marrow concentrate, said bone marrow concentrate containing said mesenchymal stem cells;
collecting blood;
centrifuging said blood to separate platelets in said blood;
concentrating said platelets to obtain a platelet rich plasma;
mixing said concentration of platelets with said concentration of bone marrow aspirate containing said bone marrow concentrate; and
using a micro needle matrix to inject said mixture of said concentration of platelets and said bone marrow concentrate into said deteriorated skin tissue;
wherein said mixture of said concentration of platelets with said concentration of bone marrow aspirate containing said bone marrow concentrate containing said mesenchymal stem cells acts to produce more fibroblasts in said deteriorated skin and stimulate the fibroblasts in said deteriorated skin.

33. The method of claim 32, wherein said bone marrow is allogenic bone marrow.

34. The method of claim 32, wherein said bone marrow is autogenous bone marrow.

35. The method of claim 34, wherein said concentration of platelets is autogenous.

36. A method for treating deteriorated skin tissue in the face or selected areas of the body of a person with mesenchymal stem cells, comprising:
obtaining an autogenous or allogeneic SVF (stromal vascular fraction) from adipose;
concentrating said SVF to obtain adipose concentrate, said adipose concentrate containing said mesenchymal stem cells; and
using a needle matrix to inject adipose concentrate into said deteriorated skin tissue;
wherein said mesenchymal stem cells improve or restore said skin tissue.

37. The method of claim 36 wherein concentrating said SVF to obtain adipose concentrate is done via centrifugation.

38. The method of claim 36 further comprising adding a premixture that includes an aqueous solution of an anticoagulant, an equal amount of dextrose, and an amount of phosphate buffered saline to said adipose concentrate to provide a delivery media formulation.

39. The method of claim 38 wherein the volume of said premixture matches or approximates the volume of said adipose concentrate.

40. The method of claim 36 further comprising making said injection of said adipose concentrate into the dermis or the hypodermis of said skin tissue.

41. The method of claim 36 further comprising making said injection of adipose concentrate within less than one hour of obtention from said person.

42. The method of claim 36 wherein the deteriorated skin tissue in the face is in the peri-orbital area, the lips, the malar area, the nasolabial folds, forehead or the labio-mandibular folds.

43. The method of claim 36 wherein the deteriorated skin tissue in the selected areas of the body is in the neck, the hands or traumatized or burned areas.

44. A method for treating deteriorated skin tissue in the face or selected areas of the body of a person with mesenchymal stem cells, comprising:
obtaining an SVF (stromal vascular fraction) from adipose;
concentrating said SVF to obtain adipose concentrate, said adipose concentrate containing said mesenchymal stem cells;
collecting blood;
centrifuging said blood to separate platelets in said blood;
concentrating said platelets to obtain a platelet rich plasma;
mixing said concentration of platelets with said adipose concentrate containing said mesenchymal stem cells; and
using a micro needle matrix to inject said mixture of a concentration of platelets and said adipose concentrate into said deteriorated skin tissue;
wherein said mixture of said concentration of platelets with adipose concentrate containing said mesenchymal stem cells acts to produce more fibroblasts in said deteriorated skin and stimulate the fibroblasts in said deteriorated skin.

45. The method of claim 44, wherein said adipose is allogenic adipose.

46. The method of claim 44, wherein said adipose is autogenous adipose.

47. The method of claim 46, wherein said concentration of platelets is autogenous.

48. An apparatus for treating skin tissue, said apparatus comprising:
a reservoir that supplies a fluid to a matrix of needles, said reservoir containing a mixture comprising mesenchymal stem cells and a platelet rich plasma
a needle matrix connected to said reservoir; and
an injector that pushes said fluid through said needle matrix.

49. The apparatus of claim 48 wherein said injector comprises a plunger and said reservoir comprises a syringe reservoir.

50. The apparatus of claim 48 wherein injector comprises a pouch and said reservoir is located inside said pouch.

51. The apparatus of claim 48 wherein said injector comprises a cylindrical roller with said reservoir inside said cylindrical roller, a surface and a plate that separates said reservoir into an empty reservoir and a fluid reservoir and wherein said cylindrical roller is connected to at least one hollow tube capable of supplying fluid to said reservoir, and wherein said micro needle matrix penetrate said surface to accept fluid from said fluid chamber.

52. The apparatus of claim 48 wherein said injector comprises a means for adjusting a depth of penetration of said matrix of needles.
